(19) United States
(12) Plant Patent Application Publication
(10) Pub. No.: US 2008/0134382 P1
(43) Pub. Date: Jun. 5, 2008

(54) Grapevine Plant Named 'Sheegene-4'

(76) Inventor: Timothy P. Sheehan, Porterville, CA (US)

Correspondence Address: Eric C. Cole;Baker, Manock & Jensen, Ste. 421, 5260 N. Palm Ave., Fresno, CA, 93704, US

(21) Appl. No.: 11/594,527

(22) Filed: Nov. 8, 2006

(51) Int. Cl. A01H 5/00

(52) U.S. Cl. PLT/205

(57) ABSTRACT

A new and distinct variety characterized by a large green nearly-seedless grape with excellent flavor that ripens in the second week of September approximately eight weeks later than the Thompson Seedless Grape (unpatented). The berries of this new variety are produced on strong woody stems and branches and are very well adapted to commercial harvesting.

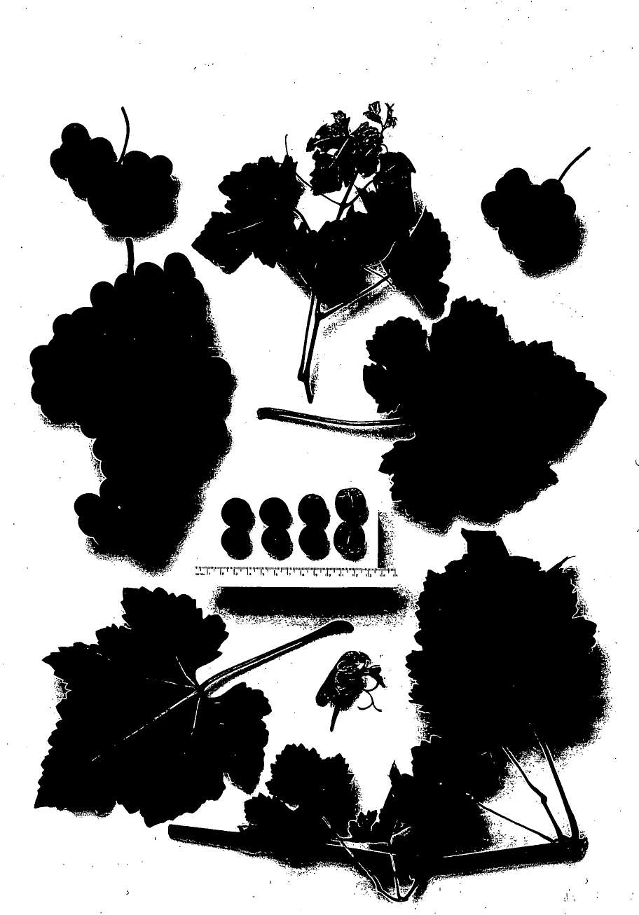

### CROSS REFERENCE TO RELATED APPLICATIONS

The claimed plant is not subject of a related application.

### STATEMENT OF ANY FEDERALLY-SPONSORED RESEARCH AND DEVELOPMENT

The claimed plant is not subject of Federally-sponsored research or development.

### LATIN NAME OF THE GENIUS AND SPECIES OF THE PLANT CLAIMED

The claimed plant relates to a new and distinct variety of Vitis vinifera to be known as Sheegene-4.

### VARIETY DENOMINATION

This invention relates to a new discovery and asexual propagation of a new variety of Vitis vinifera. The new variety was first hybridized by Timothy P. Sheehan of Porterville, Calif. in the spring of 2000; it is highly productive and produces a large green nearly-seedless grape with excellent flavor. The pollen parent is Autumn Seedless (unpatented) and the seed parent is Red Globe (U.S. Plant Pat. No. 4,787). The new variety first flowered in May 2003. The new variety of Vitis vinifera was first asexually propagated by Timothy P. Sheehan, grafted on Harmony virus-free rootstock in the dormant season of 2003/2004. Eight vines of the new variety were planted in a variety block of Vitis Vinifera in an area near Fowler, Calif. on the west side of Thompson Road north of Adams. The grapes produced by the new variety are larger than the Thompson Seedless (unpatented) and have excellent flavor. The new variety has been shown to maintain its distinguishing characteristics through asexual propagation.

### BACKGROUND OF THE INVENTION

The present invention relates to a new and distinct variety of grape which will hereinafter be denominated as Sheegene-4 and more particularly as a grapevine which produces a green nearly-seedless table grape that matures during the second week of September in the San Joaquin Valley of Central Calif. The new invention most closely resembles Thompson Seedless (unpatented) but it produces a larger grape that is very sweet taste, has excellent flavor, and matures at least eight weeks later.

### SUMMARY OF THE INVENTION

The claimed plant is characterized by producing a large green nearly-seedless grape with excellent flavor that is mature for harvesting and shipment during in the second week of September, and it can be compared to the Thompson Seedless Grape (unpatented) but that it matures about eight weeks later, among other distinguishing characteristics.

### BRIEF DESCRIPTION OF THE DRAWING

The accompanying drawing is a color photograph of the grapes produced by the subject variety; several leaves are displayed as well as a typical section of the vine of the subject variety. Across the top of the colored photograph on the left is a small cluster of berries; in the center is a shoot displaying tendrils and small leaves; and to the right is a mature leaf displaying the upper surface and petiole. Across the middle of the colored photograph on the left is a cluster of mature grapes; in the center are four grapes cut in half longitudinally as well as horizontally displaying the flesh and shape; and to the right is a shoot and tendrils including small leaves. Across the bottom of the colored photograph on the left is a mature leaf displaying the lower surface and a mature cane with small leaves displaying the nodes.

### DETAILED BOTANICAL DESCRIPTION

Referring more to the horticultural description of the new and distinct variety of grapevine, the following description has been observed under the ecological conditions prevailing at the origin vineyard that is located east of Fowler, Calif. in the San Joaquin Valley of Central Calif.

All major color designations are by reference to the Dictionary of Color by Maerz and Paul, First Edition, published in 1930. Common colors names are also used in several instances.
Vine:
   Size.—Large for a four year old vine.
   Vigor.—Very good.
   Chilling requirements.—Normal for grapes in San Joaquin Valley of Central Calif.
   Figure.—With wide cordons forming a “T” shape.
   Production capacity.—Very good.
   Regularity of bearing.—Regular.
Trunk:
   Size.—Medium — 13¼ inches (33.7 cm) in circumference, 27 inches above graft.
   Surface texture.—Rough, shaggy.
   Color of bark.—P1.7 E9 Kaffa Hamstead Brown.
   Mature cane — color.—P1.13 G10 Pekinese.
   Nodes — five on mature canes.—26 inches long (66.4 cm).
   Length between nodes.—12.7 cm (5 inches).
   Lenticel — numbers.—0.
   Lenticel — size.—0.
   Shoots — size.—Medium to large.
   Shoots — length.—3¼ to 4.5 inches (8.23 to 11.43 cm).
   Shoots — shape.—Droopy.
   Tendrils — numbers.—One at nodes.
   Tendrils — size.—Medium to large; 3¼ to 5 inches (8.23 to 12.7 cm).
   Tendrils — location.—At node.
   Tendrils form.—Bifurcate.
   Tendrils — texture.—Firm, woody.
   Buds shape.—Convex.
   Buds size.—Medium; 7 mm (0.28 inches) in length, 5 mm (0.20 inches) in width.
   Buds fruitfulness.—Very good.
Branches:
   Size.—Medium; 6.35 cm (2½ inches) circumference.
   Surface texture.—Nearly smooth.
   Cordons.—40 inches (101.6 cm) from trunk at one side, 34½ inches from trunk on other side.
   Color — one year or older wood.—P1.7 E5 Mauve Rose.
   Color — immature branches.—P1.19 L5 — Cosse gr.
Leaves:
   Size.—Generally large.
   Density.—Dense.
   Average length.—16.0 to 21.3 cm (6⅜ to 9⅛ inches).
   Average width.—18.8 to 23.5 cm (7⅞ to 10⅛ inches).
   Shape.—Pentagonal.
   Texture — upper surface.—Smooth.
   Texture — lower surface.—Glabrous.
   Color — upwardly disposed surface.—P1.23 J8 Mt. Vernon green.
   Color — lower disposed surface.—P1.21 I7 Fern.
   Color — leaf vein.—P1.17 K7 Viridine gr.
   Glandular characteristics.—0.
   Petiole size.—Large.
   Petiole length.—15.7 to 18 cm (6 to 7 inches).
   Petiole thickness.—9.8 mm (0.39 inches).
   Petiole sinus form.—Upside down U-shape.
   Lobes.—Normally four.
   Tooth size.—Medium; 19.1 to 22.2 mm (¾ to ⅞ inches) in width, 16.2 to 28.6 mm (0.638 to 1.126 inches) in length.
   Tooth — number.—Four large normally between lobes.
   Tooth — shape.—Convex.
Inflorescence:
   Size.—11.8 cm (4.65 inches).
   Number borne — per spur.—One to two.
   Number borne — per vine.—Many; 20 to 25.
Flowers:
   Flower buds — size.—Small.
   Flower buds — surface.—Glabrous.
   Flower buds — quantity.—Moderate.
   Date of bloom.—May 15, 2006.
   Date of full bloom.—May 20, 2006.
   Size of bloom.—5 to 6 mm (0.20-0.24 inches).
   Petal color.—P1.20 K6 Piquant green.
   Petal size.—Small; 1 to 2 mm (0.039 to 0.078 inches).
   Pistol color.—P1.17 L7 Viridine y.
   Date of first visible berries.—May 20, 2006.
   Size — generally.—Small; 2 mm (0.079 inches).
Fruit:
   Solids.—8.9.
   Acid.—0.38.
   Sugar/acid ratio.—49.7.
   Juice pH.—3.78.
   Seeds.—Rudimentary.
   Seed numbers.—One to two.
   Capstem pedicel.—8 mm (0.31 inches).
   Berry weight.—11.36 grams.
   Juice color.—P1.17 A6 Opaline gr.
   Size.—Large.
   Cluster — size.—Average length not including main stem; 22 to 23 cm (8.75 inches).
   Cluster — average diameter at greatest width.—12.7 cm (5 inches).
   Cluster — weight.—681.6 grams (1½ lbs.).
   Compact.—Compact.
   Cluster form.—Conical.
   Stems — generally.—Large; 6 cm (2.86 inches) in length, approximately 6 mm (0.024 inches) in width.
   Berry size.—Large.
   Berry form.—Ovate.
   Berry number.—Approximately 60 per bunch.
   Berry size — average dimension in longitudinal axis: 30 mm (1.18 inches).
   Berry size — average dimension in transverse axis.—22 mm (0.87 inches).
Skin:
   Thickness.—Thin.
   Texture.—Tough.
   Tendency to crack.—None.
   Blush color.—P1.19 K6 Sea green.
   Ground color.—Pulp: Clear.
   Lenticels.—0.
Flesh:
   Color.—P1.17 L7 Viridine y.
   Juice production.—Good.
   Flavor.—Very good.
   Aroma.—Very mild.
   Texture.—Firm.
   Ripening.—Even.
   Eating quality.—Very good to excellent.
   Resistance to disease.—Unknown.
   Harvesting.—Second week of September.
   Shipping and handling qualities.—Very good.

Having thus described and illustrated our new variety of grapevine, we claim:

1. A new and distinct variety of grapevine to be known as Sheegene-4 and the parts thereof, substantially as illustrated and described, characterized principally by its production of grapes which mature later in the season of ripening than does the Thompson Seedless Grape (unpatented) which it closely resembles, but from which it is distinguished there from by producing clusters of nearly-seedless grapes that are larger in size and sweeter in taste.
